(19) United States
(12) Patent Application Publication
(10) Pub. No.: US 2011/0207817 A1
(43) Pub. Date: Aug. 25, 2011

(54) ANTI-INFLAMMATORY COMPOUNDS AND COMPOSITIONS THEREOF

(76) Inventors: Sean M. Wetterer, Murray Hill, NJ (US); Jingjun Yang, Surrey (GB)

(21) Appl. No.: 13/127,739

(22) PCT Filed: Nov. 10, 2009
(86) PCT No.: PCT/US09/63773

§ 371 Date: May 5, 2011

Related U.S. Application Data
(60) Provisional application No. 61/112,900, filed on Nov. 10, 2008.

(51) Int. Cl. A61K 31/235; A61P 29/00

(52) U.S. Cl. 514/544

(57) ABSTRACT

This invention relates to alkyl parabens, their use as anti-inflammatory agents and compositions containing them.

### FIELD OF THE INVENTION

This invention relates to alkyl parabens, their use as anti-inflammatory agents and compositions containing them.

### BACKGROUND OF THE INVENTION

Dentures are substitutes for missing teeth and serve as a replacement for all or some of the teeth found in the oral cavity. Over time, even well fitting dentures can become ill fitting due to natural shrinkage and changes in the gum or mucosal tissues. Therefore, adherent compositions in the form of creams, liquids, powders, and liners are often used to secure dentures within the mouth. Denture adhesives usually contain water swellable polymers and/or gums in a mineral and/or petrolatum carrier. The polymers/gums hydrate upon contact with water/saliva and become tacky so that they can hold the dentures in place.

Inflammation of the oral mucosa is a frequent complaint of denture wearers. Inflammation includes swelling of the oral mucosa, redness and pain associated with swelling. Denture stomatitis (DS), which is well described in the scientific and clinical literature, is clinically characterized as inflammation of the oral mucosa, and occurs primarily in areas of the mucosa which are in contact with the denture. DS typically develops due to ill-fitting dentures or poor denture hygiene. Candida albicans is one of the species of microbes known to cause DS. In patients with stomatitis, increased density of yeast colonization and severity of inflammation often correlates with denture cleanliness and length of time between denture removal, with greater colonization and inflammation seen in patients with worse hygiene and greater continuous wear time.

Many oral care products that are marketed towards consumers with remaining natural teeth may contain ingredients for the control of bacteria in the mouth. For example, dentifrices may contain triclosan or fluoride for the reduction of cariogenic bacteria. Similarly, mouthrinses may contain alcohol, cetylpyridinium chloride, or essential oils. In rare cases, these water-based formulations may also contain preservatives to further enhance the shelf life of the product. Alkyl parabens and their salts, for example, are well known as preservatives in cosmetic and pharmaceutical formulations. Such preservatives are used only in quantities sufficient to achieve the product stability required in order to minimize formulation cost.

In contrast, most denture adhesive products are predominantly oil-based and therefore do not require comparable levels of preservative agents as other, water-based oral care products. The formulations of such products are also typically optimized for properties of adhesive and cohesive strength and therefore do not contain ingredients intended only for antimicrobial benefit. They also do not contain ingredients intended only for anti-inflammatory benefit and therefore do not satisfactorily address the symptoms of DS.

Anti-inflammatory agents, both steroidal and non-steroidal, are well known in the art. Examples include aspirin, indomethacin, corticosteroids, ibuprofen, to name a few. All of these agents claim to treat and/or prevent inflammation and pain in humans. Unfortunately, these agents are also known to cause side-effects, such as gastrointestinal disorders. Furthermore, these agents are not known for incorporation into dentifrices or denture care products since the traditional performance metric of these products has been cleaning efficacy or adhesive strength, respectively, instead of therapeutic efficacy.

U.S. Pat. No. 4,672,076 to Pittz et al., discloses benzyl salicylate, benzyl benzoate and benzyl paraben as non-steroidal anti-inflammatory agents. In particular, the patent discloses that the compounds are useful in treating pain, inflammation, swelling and other related symptoms in mammals.

U.S. Pat. No. 4,136,145 to Möller et al., discloses alkoxybenzoic acid esters as inflammation inhibitors, in particular, for topical application to skin for the protection against, and treatment of, sunburn.

WO 2008/008494 published Jan. 17, 2008, discloses a method of treating mucositis with an agent that is antifungal and antibacterial, for example, methyl parabens and/or propyl parabens.

U.S. Pat. No. 3,833,518 to Rubin et al., discloses polymeric denture adhesives with methyl and/or propyl paraben as preservatives.

Applicants have discovered that the compounds of this invention are effective anti-inflammatory agents, either alone, or in combination with at least one other anti-inflammatory agent.

### SUMMARY OF THE INVENTION

In one aspect, this invention is to a method for treating and/or preventing inflammation in a mammal, especially a human, in need thereof, said method comprising administering an effective amount of a compound of formula (I) or a pharmaceutically acceptable salt thereof:

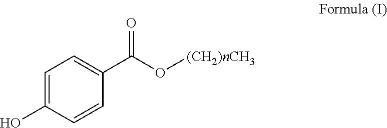

wherein:
n is an integer from zero to three.

In a second aspect, this invention is to a pharmaceutical composition comprising a compound of formula (I), at least one additional anti-inflammatory agent and a pharmaceutically acceptable excipient.

In a third aspect, this invention is to a dentifrice composition comprising a compound of formula (I) and a pharmaceutically acceptable excipient.

In a fourth aspect, this invention is to a denture adhesive composition comprising a compound of formula (I) and a pharmaceutically acceptable excipient.

In a fifth aspect, this invention is to a denture adhesive composition comprising a compound of formula (I), at least one additional anti-inflammatory agent and a pharmaceutically acceptable excipient.

In a sixth aspect, this invention is to a denture adhesive composition comprising a compound of formula (I), at least one additional anti-inflammatory agent, an anti-oxidant, and a pharmaceutically acceptable excipient.

In a seventh aspect, this invention is to a method for treating and/or preventing inflammation in an edentulous patient in need thereof, said method comprising administering a denture adhesive composition comprising an effective amount of a compound of formula (I).

In an eighth aspect, this invention is to a method for treating and/or preventing inflammation in an edentulous patient in need thereof, said method comprising administering a denture adhesive composition comprising an effective amount of a compound of formula (I) and at least one additional anti-inflammatory agent.

In a ninth aspect, this invention is to a method for treating and/or preventing inflammation in an edentulous patient in need thereof, said method comprising administering a denture adhesive composition comprising an effective amount of a compound of formula (I) and at least one anti-oxidant agent.

In a tenth aspect, this invention is to a method for treating and/or preventing inflammation in an edentulous patient in need thereof, said method comprising administering a denture adhesive composition comprising an effective amount of a compound of formula (I), at least one anti-oxidant agent and an anti-oxidant.

In an eleventh aspect, this invention is to a method of protecting the mucosal tissues of the oral cavity, in particular the gums and palette, from irritation that leads to inflammation in an edentulous patient in need thereof, said method comprising administering a denture adhesive composition comprising an effective amount of a compound of formula (I).

In a twelfth aspect, this invention is to a method of protecting the mucosal tissues of the oral cavity, in particular the gums and palette, from irritation that leads to inflammation in an edentulous patient in need thereof, said method comprising administering a denture adhesive composition comprising an effective amount of a compound of formula (I) and at least one additional anti-inflammatory agent.

In a thirteenth aspect, this invention is to a method of protecting the mucosal tissues of the oral cavity, in particular the gums and palette, from irritation that leads to inflammation in an edentulous patient in need thereof, said method comprising administering a denture adhesive composition comprising an effective amount of a compound of formula (I) and at least one anti-oxidant agent.

In a fourteenth aspect, this invention is to a method of protecting the mucosal tissues of the oral cavity, in particular the gums and palette, from irritation that leads to inflammation in an edentulous patient in need thereof, said method comprising administering a denture adhesive composition comprising an effective amount of a compound of formula (I), at least one additional anti-inflammatory agent and at least one anti-oxidant agent.

In a fifteenth aspect, this invention is to a method of treating and/or preventing the irritation associated with inflammation in an edentulous patient in need thereof, said method comprising administering a denture adhesive composition comprising an effective amount of a compound of formula (I).

In a sixteenth aspect, this invention is to a method of treating and/or preventing the irritation associated with inflammation in an edentulous patient in need thereof, said method comprising administering a denture adhesive composition comprising an effective amount of a compound of formula (I) and at least one additional anti-inflammatory agent.

In a seventeenth aspect, this invention is to a method of treating and/or preventing the irritation associated with inflammation in an edentulous patient in need thereof, said method comprising administering a denture adhesive composition comprising an effective amount of a compound of formula (I), at least one additional anti-inflammatory agent and an anti-oxidant.

### BRIEF DESCRIPTION OF THE FIGURES

FIG. 1 depicts the results of Example 4.

### DETAILED DESCRIPTION OF THE INVENTION

The term “inflammation” as used herein at all occurrences refers to the response of body tissues to injury or irritation characterized by pain and swelling and redness and heat.

The terms “preventing” or “prevention” as used herein at all occurrences refers to therapeutic therapy intended to hinder illness or injury especially that associated with inflammation.

The terms “treating” or “treatment” as used herein at all occurrences refers to therapeutic therapy intended to relieve illness or injury especially that associated with inflammation.

The term “effective amount” as used herein at all occurrences refers to the active agent(s) being present in an amount that renders the composition effective in treating and/or preventing such disease or condition.

Chemical names (IUPAC and other names) for the compounds of formula (I) and their salts are as follows: methyl 4-hydroxybenzoate; methylparaben; methyl p-hydroxybenzoate; methyl parahydroxybenzoate; 4-hydroxybenzoic acid methyl ester; methyl 4-hydroxybenzoate, sodium salt; sodium methylparaben; ethyl 4-hydroxybenzoate, ethylparaben; 4-hydroxybenzoic acid ethyl ester; ethyl paraben; ethyl p-hydroxybenzoate; ethyl parahydroxybenzoate; ethyl 4-hydroxybenzoate, sodium salt; sodium ethylparaben; propyl 4-hydroxybenzoate; propylparaben; propyl p-hydroxybenzoate; propyl parahydroxybenzoate; 4-hydroxybenzoic acid propyl ester; propyl 4-hydroxybenzoate, sodium salt; 4-hydroxybenzoic acid propyl ester, sodium salt; sodium propylparaben; butyl 4-hydroxybenzoate; butylparaben; butyl p-hydroxybenzoate; butyl parahydroxybenzoate; 4-hydroxybenzoic acid butyl ester; butyl 4-hydroxybenzoate, sodium salt; 4-hydroxybenzoic acid butyl ester, sodium salt; sodium butylparaben.

The present invention specifically relates to pharmaceutical compositions comprising, and the use of compounds of formula (I), or a pharmaceutically acceptable salt thereof, in the treatment and/or prevention of inflammation, the compound of formula (I), or a pharmaceutically acceptable salt thereof, having the following structural formula:

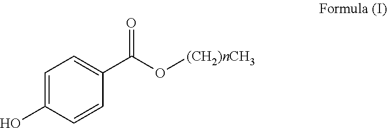

wherein:
n is an integer from zero to three.

Suitably, the compound of formula (I) is wherein n is an integer zero, which is methyl paraben.

Suitably, the compound of formula (I) is wherein n is an integer one, which is ethyl paraben.

Suitably, the compound of formula (I) is wherein n is an integer two, which is propyl paraben.

Suitably, the compound of formula (I) is wherein n is an integer three, which is butyl paraben.

While the compounds were previously known, they have not been disclosed as anti-inflammatory agents. Methyl and propyl paraben, and salts thereof, are known as preservatives in pharmaceutical formulations. (Nikitakis, J. M., 1988. CTFA Cosmetic Ingredient Handbook, first ed., The Cosmetic, Toiletry and Fragrance Association, Washington, D.C.; M. G. Soni et al. “Evaluation of the health aspects of methyl paraben: a review of the published literature” Food and Chemical Toxicology 40 (2002) 1335-1373; Soni, M. G., Burdock, G. A., Taylor, S. L., Greenberg, N. A., “Safety assessment of propyl paraben: a review of the published literature”, Food and Chemical Toxicology (2001) 39, 513-532.)

The compounds of formula (I), and pharmaceutically acceptable salts thereof, when used alone in a formulation, have been found to inhibit inflammation as measured by cellular release of the inflammatory cytokine marker, prostaglandin E2 (PGE2), when used in a therapeutically effective amount. In a clinical setting, PGE2 is associated with the manifestations of inflammation such as pain, swelling, redness and heat associated with irritation or injury to body tissues. (P. Davies, P. J. Bailey, M. M. Goldenberg and A. W. Ford-Hutchinson, “The role of arachidonic acid oxygenation products in pain and inflammation”, Annu. Rev. Immunol. 2 (1984), pp. 335-357). It has also been found that the compounds of formula (I), and pharmaceutically acceptable salts thereof, enhance the efficacy/anti-inflammatory response of known agents, for example, vitamin E (Stuyvesant, V. Wilfred; Jolley, Weldon B. “Anti-inflammatory Activity of d-α-Tocopherol (Vitamin E) and Linoleic Acid” Nature, Volume 216(5115), pp. 585-586 (1967).) and ethyl pyruvate (Fink, M. P. “Ethyl pyruvate: a novel anti-inflammatory agent”: Journal of Internal Medicine, 261(4), 2007, pp. 349-362) when used in combination. These agents are also known as anti-oxidants and/or anti-irritants.

The compounds of the invention are used in a therapeutically effective amount, and when contained in a pharmaceutically acceptable carrier or composition, are present in amount of between about 0.01 to 5.0 percent by weight of the total composition (abbreviated herein as “wt. %”). In one embodiment, the compound of formula (I) is present in an amount of between about 0.05 and 1.0 wt. %. In another embodiment, the compound of formula (I) is present in an amount of between 0.2 and 0.4 wt. %.

In one embodiment of the invention, the active agent of the pharmaceutical composition is a compound of formula (I) which is a salt of propyl paraben, namely, sodium propyl paraben.

Suitably, one or more compounds of formula (I) can be used in a formulation effective for performing the methods of this invention. In one embodiment of the invention, the active agent of the pharmaceutical composition is a combination of methyl paraben and ethyl paraben.

The compounds of the invention are typically used in a composition which can be easily and conveniently administered to a mammal, preferably, a human, experiencing symptoms of inflammation or at risk for inflammation. Dosage forms may be varied and include topical creams, pastes, ointments, gels, lotions, patches, strips, powders, and the like, for direct application to the inflamed area. Further, the compounds can be applied to an area susceptible to inflammation, such as the oral cavity, in order to prevent the symptoms of inflammation from occurring.

In one embodiment of the invention, a compound of formula (I), or a pharmaceutically acceptable salt thereof, is formulated into a denture adhesive composition. Such denture adhesive compositions are well known in the art, and include various water-soluble, water-swellable polymers, for example, polyvinyl acetate (“PVA”), polyvinyl pyrrolidone (“PVP”), or a lower alkyl vinyl ether maleic acid, anhydride, or salt copolymer or mixtures thereof, cation of the salt is selected from the group consisting of calcium, magnesium, strontium, sodium, potassium, zirconium, and zinc, or mixtures thereof; wherein the polymer is typically in an oil/wax carrier base.

U.S. Pat. No. 5,073,604 discloses a denture adhesive composition with mixed partial salts of a lower alkyl vinyl ether maleic acid copolymer, wherein said partial salts contain as the cationic salt function, (a) from about 10% to about 65% zinc or strontium cations; and (b) from about 10% to about 75% calcium cations of the total initial carboxyl groups reacted. U.S. Pat. No. 2,978,812 discloses a denture fixative composition which includes an ethylene oxide polymer having a molecular weight between 50,000 and 5,000,000 in an amount preferably comprising at least 50% of the active fixative material. GB Patent No. 1,444,485 discloses a fixing agent comprising a solution of 4 to 44 wt. % of a polyvinyl pyrrolidone. U.S. Pat. No. 3,003,988 describes the use of mixed salts of more than 40 wt. % of a water-insoluble water-sensitized polymeric material consisting essentially of lower alkyl vinyl ether maleic anhydride copolymers. U.S. Pat. No. 5,001,170 discloses a substantially anhydrous mixture of about 20-40 wt. % of methyl vinyl ether maleic acid copolymer, 20-40 wt. % of PVP, and 20-40 wt % of ethylene oxide polymer. Any one of these denture adhesive compositions would be suitable as a base for carrying the active compound of formula (I).

In addition to the foregoing materials, the denture adhesive composition may be formulated with other components well-known in the denture adhesive art including plasticizers, rheology modifiers, preservatives, humectants, emulsifiers, antioxidants, super-disintegrants or absorbents, for example, homopolymers of polyvinylpyrrolidone or copolymers of vinylpyrrolidone, flavoring agents, colorants, cross-linking agents, antimicrobial agents, control release agents, antifoaming agents, sweetening agents, viscosity modifiers and so forth.

Flavoring agents well known to the denture adhesive art may be added to the compositions of the present invention. These flavoring agents include without limitation, synthetic flavor oils and/or oils derived from plants, leaves, flowers, fruits and so forth, and combinations thereof. Representative flavor oils include, spearmint oil, cinnamon oil, oil of wintergreen (methylsalicylate) and peppermint oils. Also useful are artificial, natural or synthetic fruit flavors such as citrus oil including lemon, orange, grape, lime, and grapefruit, and fruit essences including apple, strawberry, cherry, pineapple, and so forth. The flavoring agent may be a liquid, spray dried, encapsulated, or absorbed on a carrier, and mixtures thereof. One embodiment of this invention contains as a flavoring agent, peppermint oil. The amount of flavoring agent utilized varies depending on such factors as flavor type, adhesive formulation and strength desired. In general, amounts of about 0.01 to about 5.0 wt. % of the total denture adhesive composition are suitable. In one embodiment of the invention, an amount of about 0.05 to 0.15 wt. % is used. In another embodiment, an amount of about 0.0 to about 0.1 wt. % is used.

Preservatives which may be used in the denture adhesive formulations of the invention include those known antimicrobial agents conventionally employed in the art, such as benzoic acid and sodium benzoate; sorbic acid and sorbates; propionic acid and propionates; acetic acid and acetates; nitrates and nitrites; sulfur dioxide and sulfites; antibiotics; diethyl pyrocarbonate; epoxides; hydrogen peroxide; and phosphates.

The denture adhesive compositions may also include the use of sweeteners well known in the art. The sweetening agent may be selected from a wide range of materials including water-soluble agents, water-soluble artificial sweeteners, and dipeptide based sweeteners, including mixtures thereof. Representative sweeteners include without limitation, (a) water-soluble sweetening agents such as monosaccharides, disaccharides and polysaccharides such as xylose, ribose, glucose, mannose, galactose, fructose, dextrose, sucrose, sugar, maltose, partially hydrolyzed starch, or corn syrup solids and sugar alcohols such as sorbitol, xylitol, mannitol, maltitol, hydrogenated starch hydrolysate, and mixtures thereof; (b) water-soluble artificial sweeteners such as the soluble saccharin salts, i.e., sodium or calcium saccharin salts, cyclamate salts, acesulfam-K, sucralose, and the like, and the free acid form of saccharin; and (c) dipeptide based sweeteners such as L-aspartyl-L-phenylalanine methyl ester, and the like. In general, the amount of sweetener may be about 0.001 to about 5 wt. % of the total denture adhesive composition.

The colorants useful in the present invention include the pigments such as titanium dioxide, and may also include dyes suitable for food, drug and cosmetic applications. These colorants are known as FD&C dyes. Illustrative examples include without limitation, indigo dye, known as FD&C Blue No. 2, which is the disodium salt of 5,5′-indigotindi-sulfonic acid; FD&C Green No. 1, comprising a triphenylmethylene dye and is the monosodium salt of the 4-[4-N-ethyl-p-sulfobenzylamino) diphenylmethylene]-[1-(N-ethyl-N—P-sulfobenzyl)-2,5-cyclohexadienimine]. One embodiment of the invention uses FD&C Red No. 3 as a colorant.

The viscosity modifiers useful herein include without limitation, quaternary ammonium compounds and similar agents, starches, gums, casein, gelatin and semi-synthetic cellulose.

The denture adhesive compositions may be in the form of a powder, a paste, a cream, a gel or a liner. These pastes or gels can either be applied by consumers from a container such as a tube, a brush pen, a spray bottle, a glue stick, or any other specially designed container with a consumer use friendly applicator, or can be fabricated into hydrogel films or hydrogel sheets, hydrogel strips or hydrogel wafers. These films or strips will possess a certain desirable thickness, strength and integrity during their application.

In one embodiment of the invention, a compound of formula (I), or a pharmaceutically acceptable salt thereof, is formulated in combination with at least one anti-inflammatory agent. Suitable anti-inflammatory agents include, but are not limited to, vitamins (vitamin E, vitamin B2, folic acid, etc.); NSAIDS (aspirin, ibuprofen, ketoprofen, etc.); steroidal anti-inflammatory compounds (corticosteroids); natural extracts (tumeric, green tea extract, ginger extract, etc.); biological compounds (omega-3 fatty acids, ethyl pyruvate, etc.). In a preferred embodiment, the anti-inflammatory agent is vitamin E. In general, amounts of about 0.01 to 5.00 wt. % of the total denture adhesive formulation are suitable. In another preferred embodiment, the anti-inflammatory agent is vitamin E, and is present in an amount of 0.25 wt. % of the formulation.

In one embodiment of the invention, a compound of formula (I), or a pharmaceutically acceptable salt thereof, is formulated in combination with at least one anti-oxidant agent. Suitable anti-oxidant agents include, but are not limited to, vitamins (vitamin A, vitamin C, vitamin E, etc.); biological compounds (resveratrol, EGCG, lycopene, etc.); food preservatives (TBHQ, BHA, BHT, etc); and natural extracts (soy, grape, olive oil, etc.) In general, amounts of about 0.01 to 5.00 wt % of the total denture adhesive formulation are suitable.

Means for preparing such formulations are well known in the denture adhesive art, employing conventional types of mixing equipment for blending, heating, and cooling solids and liquids. In one embodiment, the process of making a gel or paste formulation comprises the steps of: preparing a dry polymer powder mixture; preparing the medium such as water, glycerin or mixture of water/glycerin; adding the pre-made polymer powder mixture into the liquid medium and mixing until a uniform gel or paste is formed; and optionally at the end of mixing, a process such as vacuum to remove the air trapped in the product can be applied.

In the powder form, the components are admixed with flavoring agents and colorants, together with other ingredients such as non-toxic anti-caking agents (silica, magnesium stearate, talcum powder, and the like). The mixture of ingredients is thoroughly agitated or stirred to yield a generally homogenous intermixing of all components.

In the liner or layer form, the components are uniformly mixed and then coated onto a non-adhesive self supporting coating layer by any conventional coating techniques, such as by spraying (if the material is liquid or slurry or dissolved or suspended in a liquid such as water) or by sifting (if the denture adhesive is in powder form). In another embodiment, the components are admixed with the previously described preservatives, flavoring agents, colorants, sweetening agents, viscosity modifiers, and so forth. The liner is then formed by any variety of techniques known in the polymer film-forming art, including casting, calendaring, coating, and extrusion. In one embodiment to form liners, the components are first mechanically softened by a ring roller; smoothed on a hydraulic press, and die-cut as desired into denture liner shapes or other desired shapes.

To further illustrate the invention, Examples are set forth below. In these, as throughout the specification and claims, all parts and percentages are by weight and all temperatures in degrees centigrade, unless otherwise indicated.

### EXAMPLES

For Examples 1 through 3, three sets of cell culture experiments were conducted in order to establish the anti-inflammatory efficacy of sodium propyl paraben. In all cases, in-vitro testing was conducted using Human Gingival Fibroblast cells suspended in low serum media. To irritate the cells, 2 μg/mL lipopolysaccharide (LPS) derived from P. gingivalis was added to the media. Sodium propyl paraben was added either directly to the media or incorporated into an adhesive matrix which was then suspended into the media by means of a permeable plastic insert. Cellular response was quantified by the presence of Prostaglandin E₂ (PGE₂), a cytokine associated with cellular irritation and inflammation, following an incubation period. Data is reported below after normalizing the level of PGE₂ release of a negative control to “100%”.

### Example 1

### Dose Response

In-vitro testing was conducted using Human Gingival Fibroblast (HGF) cells that had been purchased from ScienCell (Carlsbad, Calif.). Prior to use, the cells had been grown in a 37° C. incubator with 5% CO₂ and 95% humidity. Cells were maintained in high glucose Dulbecco's Modified Eagle's Medium (Mediatech, Manassas, Va.) with a 10% addition of heat inactivated Fetal Bovine Serum (Mediatech) and a 1% addition of Penicillin-Streptomycin 100× solution (Mediatech). Cells that had been previously frozen for long-term storage were allowed to recover/expand for at least one passage before being utilized for experimentation.

To plate the cells, the maintenance medium was aspirated and the cells were washed with Hank's Balanced Salt Solution (Mediatech). Subsequently, the HBSS was aspirated and a trypsin-EDTA solution (Mediatech) was added to ensure that the cells released from the tissue culture plastic. Maintenance medium was reapplied and fractions of the solution containing the cells were transferred to a multi-well plate. After transfer, incubation was conducted to expand the cell population to 80% confluency.

When the desired level of confluency was achieved, the maintenance medium was aspirated and one of several test solutions was added. For a negative control, a low serum medium was prepared that contained 0.5% of Fetal Bovine Serum (Mediatech) and 581.08 mg/L of L-gluatmine powder (Sigma-Aldrich, Milwaukee, Wis.) in DMEM High Glucose Media without Phenol Red and L-Glutamine (VWR Scientific, West Chester, Pa.). Low serum media were also prepared with the addition of 1, 10, or, 100 μg/mL of sodium propyl paraben (Sigma-Aldrich). The test solutions were added individually to test wells containing cells and allowed to incubate for 6 hours.

After incubation, the equivalent of 2 μg/mL of lipopolysaccharide (LPS) derived from P. gingivalis (Invivogen, San Diego, Calif.) was added to half of the test wells. After 2 hours incubation in the presence of LPS, all of the test solutions were replaced with maintenance media that was free of phenol red and had been modified to contain the same concentration of sodium propyl paraben as the test solutions. The cells were then returned to the incubator and allowed to recover for 18 hours.

Prostaglandin E₂ was chosen as a marker of cellular inflammation. An ELISA kit from R&D Systems (Minneapolis, Minn.) was used to quantify the release of PGE₂ from the cells. Supernatants were collected from the wells and centrifuged at 10000 rpm for 15 minutes prior to testing following the directions of the assay kit. Concurrently, either a MTS cell viability assay (Promega, Madison, Wis.) or a Protein DC assay (Bio-Rad, Hercules Calif.) was conducted on the cells remaining in the wells. The quantity of PGE₂ reported by the assay was normalized by the percentage of viable cells or total protein. Further normalization was conducted to assign a value of 100% to the PGE₂ release observed from the negative control test solution.

Within the concentration range examined, sodium propyl paraben was observed to be efficacious. Results are shown below in Table 1.

TABLE 1
Anti-inflammatory efficacy of sodium propyl paraben in neat solution

| | PGE₂ Release | PGE₂ Release |
|---|---|---|
| Formulation | (without LPS) | (with LPS) |
| Media Only (Control) | 100 ± 38 | 508 ± 21 |
| 1 μg/mL Sodium Propyl Paraben | — | 124 ± 52 |
| 10 μg/mL Sodium Propyl Paraben | — | 103 ± 23 |
| 100 μg/mL Sodium Propyl Paraben | 62 ± 37 | 84 ± 62 |

### Example 2

### Efficacy in Adhesive

In order to test the efficacy of sodium propyl paraben in a denture adhesive formulation, cells were transferred into a cell culture insert (BD Lifescience, Franklin Lakes, N.J.) that was designed to fit within the wells of a 24-well plate. 0.25 gram of denture adhesive (containing either 0.0% or 0.2% sodium propyl paraben) was then placed in the base of the wells. Due to the high level of water absorbency of the denture adhesive formulation, the adhesives were allowed to fully hydrate using low serum media prior to placement of the insert into the wellplate. Labile ingredients from the denture adhesive could then cross the permeable membrane of the insert to interact with the cells

The denture adhesive base used for this experiment (Adhesive Base 1) is closely related in polymeric composition and rheological properties to commercially available denture adhesive products. Major ingredients include carboxymethylcellulose, methylvinylether/maleic acid copolymer (Gantrez®), petrolatum, and mineral oil.

In this study, PGE₂ release was quantified using an ELISA kit from R&D Systems and normalized with a MTS Cell Viability Assay kit from Promega. Further normalization was conducted to assign a value of 100% to the PGE₂ release observed from an adhesive formulation without sodium propyl paraben.

Even when delivered from a polymeric matrix, sodium propyl paraben was observed to be efficacious. Results are shown below in Table 2.

TABLE 2
Anti-inflammatory efficacy of sodium propyl paraben in a
denture adhesive

| | PGE₂ Release | PGE₂ Release |
|---|---|---|
| Formulation | (without LPS) | (with LPS) |
| Adhesive Base 1 | 100 ± 17 | 177 ± 16 |
| Adhesive Base 1 + 0.2% | 31 ± 5.4 | 17 ± 1.0 |
| Sodium Propyl Paraben | | |

### Example 3

### Efficacy in a Complex Adhesive

In this study, 0.2% w/w sodium propyl paraben was added to prototype denture adhesive formulations (Adhesive Base 2, Adhesive Base 3) that are closely related in polymeric composition and rheological properties to commercially available denture adhesive products. As with Adhesive Base 1, major ingredients include carboxymethyl-cellulose, methylvinylether/maleic acid copolymer (Gantrez®), petrolatum, and mineral oil. In some experiments, an additional package of active ingredients with anti-inflammatory efficacy was also added to the adhesive bases.

In this study, PGE₂ release was quantified using an ELISA kit from R&D Systems and normalized with a Protein DC Assay kit from Bio-Rad. Further normalization was conducted to assign a value of 100% to the PGE₂ release observed from the adhesive bases without sodium propyl paraben or any other additional actives.

Even when delivered from a denture adhesive base in the presence of anti-inflammatory actives, sodium propyl paraben was observed to be efficacious. Results are shown below in Table 3.

TABLE 3
Anti-inflammatory efficacy of sodium propyl paraben in a
complex adhesive

| | PGE₂ Release | PGE₂ Release |
|---|---|---|
| Formulation | (without LPS) | (with LPS) |
| Adhesive Base 2 | 100 ± 45 | 421 ± 83 |
| Adhesive Base 2 + Actives | 73 ± 43 | 54 ± 4.8 |
| Adhesive Base 2 + Actives + | 18 ± 6.0 | 27 ± 0.5 |
| 0.2% Sodium Propyl Paraben | | |
| Adhesive Base 3 | 100 ± 19 | 173 ± 9.4 |
| Adhesive Base 3 + Actives | 71 ± 1.2 | 109 ± 30 |
| Adhesive Base 3 + Actives + | 7.4 ± 1.9 | 40 ± 12 |
| 0.2% Sodium Propyl Paraben | | |

### Example 4

### Study of the Effects of Sodium Propyl Paraben on Gingival Inflammation Induced by Lipopolysaccharide (LPS) Obtained from Porphyromonas gingivalis

In the presence and absence of sodium propyl paraben, mouse macrophage cells (RAW 264.7) and human gingival cells (HGF) were stimulated with LPS and the levels of inflammatory mediators Prostaglandin E₂ (PGE₂) and interleukin 8 (IL-8) in the cell supernatants were measured. While LPS administration increased the secretion of inflammatory mediators, sodium propyl paraben decreased LPS induced secretion of these mediators in a dose dependent manner. Sodium propyl paraben was identified as a novel anti-inflammatory agent, inhibiting the secretion of inflammatory mediators from both leukocytes and gingival fibroblasts.

Leukocytes and gingival fibroblasts are two cell types intimately involved in gum inflammation PGE₂ and IL-8 are mediators that play a crucial role in gum inflammation. This study investigated the effect of sodium propyl paraben on the secretion of inflammation mediators from P. gingivalis LPS induced cellular models.

Mouse macrophage Raw 264.7 cells and human gingival fibroblast HGF cells were purchased from ATCC (Manassas, Va.) and ScienCell LLC (Carlsbad, Calif.), respectively. All of the culturing reagents were purchased from Mediatech (Manassas, Va.) unless otherwise stated. Both cell lines were cultured in a humidified incubator at 37° C. and in the presence of 5% CO₂ and maintained in Dulbecco's Modified Eagle Medium (DMEM, 4.5 g/l glucose) supplemented with 10% Fetal Bovine Serum (FBS) and 1% penicillin-streptomycin. Prior to cell treatment, the maintenance media were replaced with penicillin-streptomycin free/phenol-red free DMEM media to avoid possible interference with LPS and assay reagents.

Cells were cultured in 24 or 48 well plates overnight to reach 50-70% confluency. Low serum media (0.5% FBS) were prepared with the addition of 1, 10, or 100 μg/ml of sodium propyl paraben (Sigma-Aldrich, St. Louis, Mo.). The test solutions were added individually to test wells containing cells and allowed to incubate for 6 hours. Following incubation, P. gingivalis LPS (PG-LPS) at 2 μg/ml was introduced to designated test wells. After 2 hours incubation in presence of LPS, all of the media were replaced with maintenance media (10% FBS) that contain the same concentration of sodium propyl paraben. The cells were further incubated for 18 hours prior to sample collection and assaying.

### Cell Viability Assay

Cells were incubated with media containing 1, 10, or 100 μg/ml of sodium propyl paraben for 24 hours. Potential cell toxicity of the agents was determined by a cell viability/proliferation assay using CellTiter 96 Aqueous One Solution Cell Proliferation Assay Kit (Promega, Madison, Wis.) following manufacturer's instructions.

### Inflammation Markers Analysis

Supernatants were collected and centrifuged at 10,000 rpm for 5 minutes. The level of PGE₂ or IL-8 was analyzed using commercially available enzyme-linked immunosorbent assay (ELISA) kits (R&D Systems, Minneapolis, Minn.). The results were normalized by cell viability of the same sample and normalized results were compared with LPS (+) and (−) control groups.

### Statistical Methods

Data are reported as means (+/−Standard Deviation). Comparison was made between LPS control and sample treated with sodium propyl paraben and LPS. Student's t-test was used to determine the statistical significance of the findings. Differences were considered significant when p<0.05

### Results

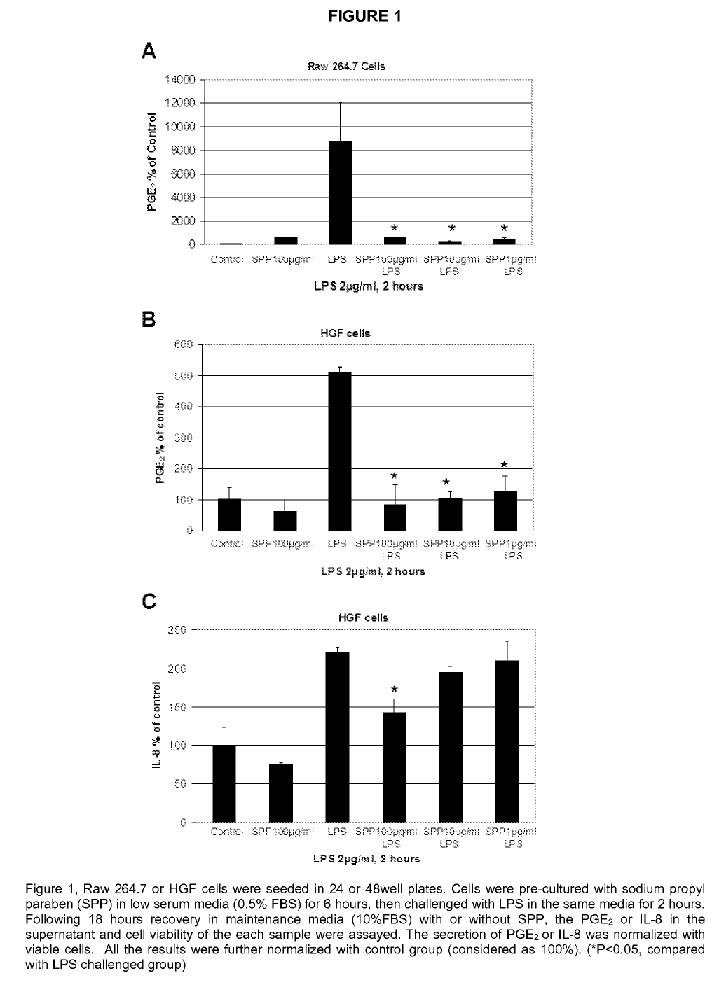

LPS induced a greater release of PGE₂ from Raw 264.7 cells compared to the negative control. The elevated release of PGE₂ was reduced to basal level by sodium propyl paraben at 1, 10 and 100 μg/ml (See, FIG. 1A), indicating a strong anti-inflammatory effect. To further investigate the effect of sodium propyl paraben on local gingival inflammation, HGF cells were treated the same way with LPS and sodium propyl paraben. LPS treatment resulted in a 5-fold increase in PGE₂ release relative to the control. The ability of sodium propyl paraben to inhibit LPS mediated PGE₂ release in HGF was similar to the inhibition observed in Raw 264.7 cells (See, FIG. 1B). In a parallel experiment, sodium propyl paraben reduced LPS mediated IL-8 production in a dose dependent manner (See, FIG. 1C). Induction of PGE2 or IL-8 was not observed upon treatment with propyl paraben at 100 μg/ml in either cell model.

The following formulations were made and are included within the scope of this invention.

Denture Adhesive Formulation 1

| | Ingredients | % w/w |
|---|---|---|
| 1 | Mixed Calcium and Sodium salt of | 30.000 |
| | methylvinylether/maleic acid copolymer | |
| 2 | Petrolatum Blend | 27.900 |
| 3 | Carboxymethylcellulose | 24.000 |
| 4 | Mineral Oil | 16.575 |
| 5 | Ethyl Pyruvate | 0.500 |
| 6 | d-alpha-Tocopherol Acetate | 0.250 |
| 7 | Sodium Propyl Paraben | 0.400 |
| 8 | Undecylenic Acid | 0.100 |
| 9 | l-Menthyl Lactate | 0.125 |
| 10 | Menthol | 0.100 |
| 11 | FD & C Red No. 7 Lake Paste | 0.020 |
| 12 | FD & C Red No. 30 Lake Paste | 0.030 |
| | Total | 100.000 |

Denture Adhesive Formulation 2

| | Ingredients | % w/w |
|---|---|---|
| 1 | Mixed Zinc, Magnesium, and Sodium salt of | 30.000 |
| | methylvinylether/maleic acid copolymer | |
| 2 | Petrolatum Blend | 27.000 |
| 3 | Carboxymethylcellulose | 24.000 |
| 4 | Mineral Oil | 15.675 |
| 5 | Silicon Dioxide | 1.100 |
| 6 | Methylvinylether/maleic acid copolymer | 1.000 |
| 7 | Ethyl Pyruvate | 0.500 |
| 8 | d-alpha-Tocopherol Acetate | 0.250 |
| 9 | Sodium Propyl Paraben | 0.200 |
| 10 | l-Menthyl Lactate | 0.125 |
| 11 | Menthol | 0.100 |
| 12 | FD & C Red No. 7 Lake Paste | 0.020 |
| 13 | FD & C Red No. 30 Lake Paste | 0.030 |
| | Total | 100.000 |

Formulation 3

| | Ingredients | % w/w |
|---|---|---|
| 1 | Polyvinyl Acetate | 73.750 |
| 2 | Ethyl Pyruvate | 0.500 |
| 3 | d-alpha-Tocopherol Acetate | 0.250 |
| 4 | Sodium Propyl Paraben | 0.400 |
| 5 | Undecylenic Acid | 0.100 |
| 6 | Ethanol | 25.000 |
| | Total | 100.000 |

Formulation 4

| | Ingredients | % w/w |
|---|---|---|
| 1 | Polyvinyl Acetate | 62.750 |
| 2 | Triacetin | 18.000 |
| 3 | Ethyl Pyruvate | 0.500 |
| 4 | d-alpha-Tocopherol Acetate | 0.250 |
| 5 | Sodium Propyl Paraben | 0.400 |
| 6 | Undecylenic Acid | 0.100 |
| 7 | Ethanol | 18.000 |
| | Total | 100.000 |

Formulation 5

| | Ingredients | % w/w |
|---|---|---|
| 1 | Polyvinyl Acetate | 51.250 |
| 2 | Ethyl Pyruvate | 0.500 |
| 3 | d-alpha-Tocopherol Acetate | 0.250 |
| 4 | Sodium Propyl Paraben | 0.400 |
| 5 | Undecylenic Acid | 0.100 |
| 6 | Water | 27.200 |
| 7 | Ethanol | 20.300 |
| | Total | 100.000 |

Formulation 6

| | Ingredients | % w/w |
|---|---|---|
| 1 | Polyvinyl Acetate | 58.800 |
| 2 | Polyethylene glycol 400 | 18.000 |
| 3 | Triacetin | 5.000 |
| 4 | Silicon Dioxide | 1.950 |
| 5 | Ethyl Pyruvate | 0.500 |
| 6 | d-alpha-Tocopherol Acetate | 0.250 |
| 7 | Sodium Propyl Paraben | 0.400 |
| 8 | Undecylenic Acid | 0.100 |
| 9 | Flavor | 0.790 |
| 10 | Saccharine | 0.210 |
| 11 | Silicone Oil | 4.000 |
| 12 | Ethanol | 10.000 |
| | Total | 100.000 |

Denture Adhesive Formulation 7

| | Ingredients | % w/w |
|---|---|---|
| 1 | Mixed Calcium and Sodium salt of | 30.00 |
| | methylvinylether/maleic acid copolymer | |
| 2 | Petrolatum Blend | 27.75 |
| 3 | Carboxymethylcellulose | 24.00 |
| 4 | Mineral Oil | 17.62 |
| 5 | Methyl paraben | 0.20 |
| 6 | d-alpha-Tocopherol Acetate | 0.25 |
| 7 | Ethyl paraben | 0.18 |
| | Total | 100.00 |

It should be understood that the foregoing description is only illustrative of the present invention. Various alternatives and modifications can be devised by those skilled in the art without departing from the present invention. Accordingly, the present invention is intended to embrace all such alternatives, modifications and variations that fall within the scope of the appended claims.

1. A method for treating and/or preventing inflammation in a mammal, especially a human, in need thereof, said method comprising administering an effective amount of a compound of formula (I) or a pharmaceutically acceptable salt thereof: 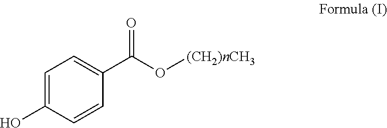
wherein:
n is an integer from zero to three.

2. A method for treating and/or preventing inflammation in a mammal, especially a human, in need thereof, said method comprising administering a pharmaceutical composition comprising an effective amount of a compound of formula (I), or a pharmaceutically acceptable salt thereof: 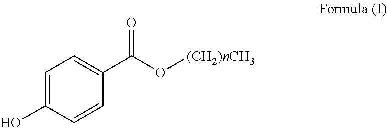
wherein:
n is an integer from zero to three, and a pharmaceutically acceptable excipient.

3. The method as claimed in claim 2, wherein the pharmaceutical composition further comprising at least one anti-inflammatory agent.

4. The method as claimed in claim 2, wherein the pharmaceutical composition further comprising at least one anti-oxidant agent.

5. A method for treating and/or preventing inflammation in a mammal, especially a human, in need thereof, said method comprising administering a dentifrice composition comprising an effective amount of a compound of formula (I) or a pharmaceutically acceptable salt thereof: 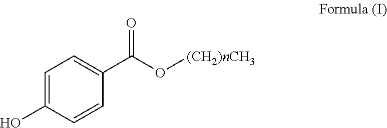
wherein:
n is an integer from zero to three, and a pharmaceutically acceptable excipient.

6. The method as claimed in claim 5, wherein the dentifrice composition further comprising at least one anti-inflammatory agent.

7. The method as claimed in claim 5, wherein the dentifrice composition further comprising at least one anti-oxidant agent.

8. A method for treating and/or preventing inflammation in a human in need thereof, said method comprising administering a denture adhesive composition comprising an effective amount of a compound of formula (I) or a pharmaceutically acceptable salt thereof: 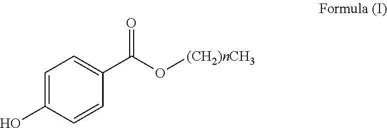
wherein:
n is an integer from zero to three, and a pharmaceutically acceptable excipient.

9. The method as claimed in claim 8, wherein the denture adhesive composition further comprising at least one anti-inflammatory agent.

10. The method as claimed in claim 8, wherein the denture adhesive composition further comprising at least one anti-oxidant agent.

11. A method for treating and/or preventing inflammation in an edentulous patient in need thereof, said method comprising administering a denture adhesive composition comprising an effective amount of a compound of formula (I) or a pharmaceutically acceptable salt thereof: 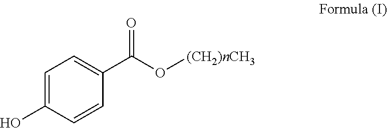
wherein:
n is an integer from zero to three.

12. A method for treating and/or preventing inflammation in an edentulous patient in need thereof, said method comprising administering a denture adhesive composition comprising an effective amount of a compound of formula (I) or a pharmaceutically acceptable salt thereof: 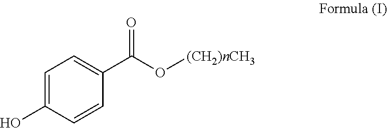
wherein:
n is an integer from zero to three, and at least one additional anti-inflammatory agent.

13. A method for treating and/or preventing inflammation in an edentulous patient in need thereof, said method comprising administering a denture adhesive composition comprising an effective amount of a compound of formula (I) or a pharmaceutically acceptable salt thereof: 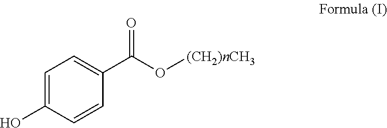
wherein:
n is an integer from zero to three, and at least one anti-oxidant agent.

14. A method for treating and/or preventing inflammation in a mammal, especially a human, in need thereof, said method comprising administering a pharmaceutical composition comprising an effective amount of a compound of formula (I), or a pharmaceutically acceptable salt thereof: 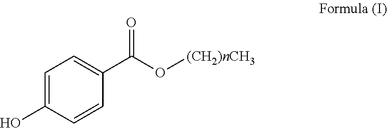
wherein:
n is an integer from zero to three, at least one additional anti-inflammatory agent, at least one anti-oxidant agent, an antimicrobial agent and a pharmaceutically acceptable excipient.
